(19) United States
(12) Patent Application Publication
(10) Pub. No.: US 2003/0120244 A1
(43) Pub. Date: Jun. 26, 2003

(54) Novel leg cuff configuration

(76) Inventor: Betsy Johnson, Atlanta, GA (US)

Correspondence Address: Christopher C. Campbell, Esq., Hunton & Williams, Suite 1200, 1900 K Street, NW, Washington, DC, 20006-1109, US

(21) Appl. No.: 10/024,493

(22) Filed: Dec. 21, 2001

(51) Int. Cl. A61F 13/15; A61F 13/20

(52) U.S. Cl. 604/385.27; 604/385.23

(57) ABSTRACT

Absorbent articles having a novel leg cuff configuration, methods for using same and methods for preparing same, are disclosed. In particular, the absorbent articles comprise a polymer barrier and a pair of leg cuffs configured to provide unexpectedly superior comfort for wearers of the absorbent articles. The absorbent articles further unexpectedly provide superior fit about the legs of the wearer and consequentially superior lateral leak protection.

### FIELD OF THE INVENTION

The present invention relates generally to the field of absorbent articles, and more particularly to absorbent articles having a novel leg cuff configuration, methods for using same and methods for preparing same. In particular, the present invention relates to absorbent articles having unexpectedly superior fit, distribution, comfort, leak protection, skin protection, absorbency and/or cost-efficiency, and methods for using same and methods for preparing same.

### BACKGROUND OF THE INVENTION

Disposable absorbent articles typically include a moisture-impervious backing sheet, an absorbent pad, and a liner sheet that contacts the body of a person wearing the article. In addition, elasticized regions are provided around the edges of the article to secure the article about the waist and legs of a wearer. Absorbent articles, such as disposable diapers, typically further comprise opposed front and rear waist portions defining a waist opening, a crotch portion disposed therebetween, and a pair of elastically contractible leg openings along the side edges of the crotch portion. Disposable diapers having elasticized margins for placement about the legs of a wearer are disclosed in U.S. Pat. No. 4,050,462 and U.S. Pat. No. 5,092,861, and disposable diapers having elasticized side margins and waist band margins are disclosed in U.S. Pat. No. 4,300,562. Further, disposable diapers are conventionally fastened to the body of a user using various attachment means, including adhesive tapes, velcro tapes, spring-like clasps and flaps.

Problems with such diaper designs include shifting, shearing and conflicting movements of the article and the body of a wearer, which distort the article, and thereby cause uneven distribution of the article relative to the wearer's body and/or cause the article to move away from a position in which it is able to reliably and intimately contact the wearer's body. Such distortions cause discomfort and often result in lateral leakage of urinary or fecal material from the diaper. Further, prolonged contact of liquid or semi-solid excreta with the skin of the wearer is also aggravated under such circumstances. For example, the moisture vapor and heat generated by the bodily exudate accumulate in a pocket of the diaper, becoming trapped and then leading to conditions adjacent to a wearer's skin that promote skin irritation, infection, and the like. Although a plastic backsheet, as described above, is generally effective in precluding the passage of bodily exude outwardly, the backsheet is not efficient in preventing lateral leakage of liquids from the opposed side portions of the core sideward between the leg gathers of the backsheet and the wearer's skin. One solution to this problem, the tightening of leg gathers or leg cuffs, presents problems in terms of the comfort of the baby and further skin irritation, particularly in view of the conventional use of polymer linings on the leg cuffs.

Despite previous advancements in the field of absorbent articles, persons of ordinary skill in the art continue their efforts to produce garments that fit better and thus are more comfortable and better able to contain urinary and fecal excretions. Various approaches have been attempted to improve fit and comfort of absorbent articles, while reducing lateral leakage of liquids from such articles. However, these approaches are all deficient in terms of effectiveness and/or cost-efficiency.

For example, U.S. Pat. No. 6,114,596 to Nayak et al. discloses a breathable diaper, feminine hygiene, or like disposable sanitary product having a cloth-like outer surface and including a plurality of materials from the skin-facing side outwardly, a topsheet, a core, an optional barrier, and a backsheet. The disclosed topsheet is formed of liquid- and vapor-permeable hydrophilic material, and the core is formed of highly absorbent material disposed outwardly of the topsheet for absorbing liquid received through the topsheet. The disclosed core has an inter surface in liquid communication with the topsheet and an outer surface. The disclosed optional barrier is formed for a multi-layer non-woven material which is hydrophobic and vapor-permeable for limiting the outward escape of liquid therethrough while enabling the outward escape of heat and water vapor therethrough. The disclosed barrier has a base disposed adjacent the core outer surface. The disclosed backsheet is formed of a multi-layer non-woven material which is hydrophobic and vapor permeable for limiting the outward escape of liquid therethrough while enabling the outward escape of heat and water vapor therethrough. This backsheet is disclosed as being disposed at least partially as an outer surface of the diaper.

U.S. Pat. No. 6,017,336 to Sauer discloses an absorbent article which includes a pair of compression resistant containment barriers which are configured to inhibit the lateral flow of fecal exudates along the surface of the absorbent article. The containment barriers are disclosed as being laterally spaced apart to provide a void space between the wearer's back side and the surface of the absorbent article for containing body exudates. Each containment barrier is disclosed as defining a width to height ratio of at least about 0.5 and a compression resistance of at least about 50%. The absorbent article is disclosed as optionally including a containment dam which is located on the body facing surface of the absorbent article and which is configured to inhibit a longitudinal flow of fecal exudates along the surface of the absorbent article.

U.S. Pat. No. 5,843,066 discloses a disposable absorbent article having a breathable laminate backsheet. The absorbent article has a front waist region, a rear waste region, a crotch region positioned between the front waist region and the rear waste region, a pair of opposed side edges, a first end edge and a second end edge. The absorbent article includes an absorbent core having a pair of opposing longitudinal edges, a garment-facing side of the absorbent core. The breathable laminate backsheet has an inner layer and an outer layer joined to one another. The inner layer is positioned between the absorbent core and the outer layer. The outer layer is relatively vapor pervious. The inner layer is substantially liquid, vapor and air impermeable. The inner layer is longitudinally coterminous with the absorbent article and of a smaller transverse width than the front waist region, the rear waste region, and the crotch region so as to form a breathable zone which is longitudinally coterminous with the absorbent article and adjacent to each of the side edges.

U.S. Pat. No. 5,597,906 to Roe, et al. discloses an absorbent article comprising a liquid pervious topsheet, a liquid pervious backsheet joined to at least a portion of the topsheet, an absorbent cord disposed between at least a portion of the topsheet and the backsheet, and a waste management element disposed in at least a portion of the crotch region. The waste management element preferably has an acceptance under pressure value of greater than about 0.50 grams of a viscous fluid bodily waste per square inch of the waste management element milliJoule of energy input. The waste management element is also disclosed as having a storage under pressure values of at least about 0.70 grams of the viscous fluid bodily waste per square inch of the waste management element. The waste management element is also disclosed as optional having an Immobilization Under Compressed Inversion valve of greater than about 70% of the viscous fluid bodily waste accepted by the waste management element. The waste management is disclosed as being located anywhere in the article, including the crotch region.

U.S. Pat. No. 5,941,864 to Roe discloses a disposable absorbent article, such as a diaper having a first topsheet with apertures large enough for low-viscosity fecal material to pass through to a fecal material storage element. The fecal material storage element is disclosed as immobilizing the fecal material in position for dewatering, so that the liquid components of the fecal material are absorbed by the core and solid components of the fecal material are separated from the liquid components, to provide for easier cleaning of the wearer when the soiled disposable absorbent article is removed.

U.S. Pat. No. 5,941,862 to Haynes et al. discloses absorbent structures comprising a first layer, a second layer juxtaposed in facing relation with said first layer, wherein at least one of the layers is fluid pervious. These structures are disclosed as having a continuous region between said first and second layers comprising hydrogel-forming polymer that is substantially uniformly distributed throughout the region. The continuous region is disclosed as at least partially surrounding multiple, spaced apart zones between said layers, which zones are substantially devoid of hydrogel-forming absorbent polymer. The first and second layers are disclosed as being bonded together such that said hydrogel-forming absorbent polymer is substantially immobilized when in dry state, an preferably at sites within plurality of the zones.

U.S. Pat. No. 5,558,655 to Jezzi et al. discloses in absorbent article, such as diaper, which comprises a composite structure of a “two-dimensional” or very flat, apertured film or nonwoven layer, in combination with a fluid transferring layer and a superabsorbent polymer-containing laminate, and a absorbent core below the laminate to achieve superior dryness. The superabsorbent polymer-containing laminate is disclosed as containing airlaid fibrous components and superabsorbent polymers and as swelling to at least three times its dry caliper upon fluid introduction in order to achieve movement of the coverstock away from the absorbent core which permits the coverstock to remain relatively dry and avoids rewetting.

U.S. Pat. No. 6,040,251 to Caldwell discloses barrier webs at a certain desirable physical qualities such as water resistance, increased durability, improved barrier qualities and the like. Barrier webs are disclosed as comprising a web that has been treated with a curable shear thinned thixotropic polymer composition which is adapted to be substantially impermeable to liquids, permeable to gases and impermeable to microorganisms. Further, the barrier webs are disclosed as being either impermeable to all microorganisms or impermeable to microorganisms of certain sizes. Also disclosed are fabrics that are capable of either selectively binding certain microorganisms, particles, or molecules depending upon what binding partners are incorporated into the polymer before application to the fabric.

U.S. Pat. No. 5,945,175 to Yahiaoui et al discloses a coated porous substrate composed of a hydrophobic polymer which is substantially uniformly coated with a hydrophilic polymeric material. The substrate may be a sheet-like material, examples of which are forms, fibers, and fibrous webs. The fibrous webs are disclosed as desirably being nonwoven webs. The coating on the substrate is disclosed as being durable to an aqueous medium at a temperature in a range of from about 10° C. to about 50° C. and does not significantly suppress the surface tension of an aqueous medium with which the coated substrate may come in contract. The hydrophobic polymer is disclosed as being a polyolefin, such as polyethylene or polypropylene. The hydrophilic polymeric material is disclosed as being a polysaccharide or a modified polysaccharide.

U.S. Pat. No. 6,117,121 to Faulks et al. discloses an absorbent article including an absorbent core located between a bodyside liner and an outer cover. The absorbent article is disclosed as having a leg cuff mounted to a base structure in the crotch portion thereof. The leg cuffs are disclosed as being partially stretched when attached to respective longitudinal side portions near the crotch portion of the absorbent article.

U.S. Pat. No. 6,107,539 to Palumbo et al. discloses disposable absorbent articles comprising a backsheet, a topsheet, a fluid acquisition/distribution region and at least one fluid storage region, said article having a total product acquisition performance of more than 3.75 ml/sec in the first gush and more than 0.5 ml/sec in the fourth gush and an in bag stack height of less than 9.9 mm, characterized in that said topsheet allows it to retain no more then 0.25 g of fluid as measured by the topsheet-on-acquisition-material-wetness test, and that said acquisition/distribution region has a drip capacity of at least 5.0 grams of fluid per gram of material.

U.S. Pat. No. 5,935,118 to Gryskiewicz et al. discloses an absorbent article that includes a garment shell and at least one liquid containment beam formed of an absorbent material. The liquid containment beam has an attachment edge bonded to the garment shell so that the containment beam can lie against the garment shell and also pivot about an axis defined by the attachment edge. For comfort during use, the containment beam desirably has a width to thickness ratio of at least about 3:1. In particular embodiments, the absorbent article includes pairs of inner and outer containment beams, with the inner containment beams adapted to lie against the garment shell and the outer containment beams adapted to lie against the inner containment beams.

U.S. Pat. No. 6,087,550 to Anderson-Fischer et al. relates to water responsive thermoplastic compositions and articles constructed thereof. This invention particularly relates to thermoplastic copolyester compositions useful for the manufacture of disposable articles such as disposable diapers and feminine napkins. More particularly, this invention relates to thermoplastic copolyester compositions that are useful as a raw material in the manufacture of nonwovens, barrier films or coatings, as well as for various improved hot melt adhesive compositions useful for incorporating hydrophilic features into disposable articles.

U.S. Pat. No. 6,120,783 to Roe et al. discloses web materials which have two or more skin care compositions disposed thereon. The skin care compositions are transferable to the wearer's skin by normal contact and/or wearer motion and/or body heat. The skin care compositions disclosed in the present invention are selected to maintain and/or improve the skin health of the wearer upon transfer during use, for example, to provide a skin protective barrier or a therapeutic benefit; to minimize the abrasion between the cuffs and skin in the area where the cuffs contact the wearer's skin, resulting in less skin irritation; to improve BM clean up on the skin, or to improve the barrier properties of the cuffs. Web materials of the present invention have a wide range of potential uses in both durable and disposable articles, but are particularly well suited for use in disposable absorbent articles such as disposable diapers, incontinent briefs, training pants, sanitary napkins, and the like.

U.S. Pat. No. 5,683,809 to Freeman et al. discloses protective articles such as diapers, having filmless hydrophobic barrier elements such as cuffs and backing sheets. The barrier cuffs—which can be, for instance, leg cuffs and waistbands—and the backing sheets can be provided from fabrics having a fabric weight of at least 10 gsy.

U.S. Pat. No. 5,928,209 to Bodford et al. discloses a breathable diaper, feminine hygiene, or like disposable sanitary product construction includes a plurality of materials including, from the skin-facing side outwardly, a topsheet of liquid- and vapor-permeable hydrophilic material. A core of highly absorbent material is disposed outwardly of the topsheet for absorbing fluid received through the topsheet, the core having an inner surface in fluid communication with the topsheet. A backsheet is disposed at least partially as an outer surface of the construction and is formed of a pouch defined by two layers of a non-woven hydrophobic and vapor-permeable material, with absorbent or superabsorbent particles therebetween, so that the backsheet limits the outward escape of liquid therethrough while enabling the outward escape of heat and water vapor therethrough. Each backsheet layer is an at least two-layer spunbound-meltblown SM or an at least three-layer spunbond-meltblown-spunbond SMS.

U.S. Pat. No. 5,990,377 to Chen et al. discloses a dual-zoned, three-dimensional, resilient absorbent web is disclosed which is suitable as body-side liner for absorbent articles such as feminine pads, diapers and the like. When used as a liner in absorbent articles, the dual-zoned web combines the advantages of apertured films and soft, nonwoven cover layers in one structure while still being inherently hydrophilic. The liner comprises a web of wet-resilient, hydrophilic basesheet having a three-dimensional topography comprising elevated regions onto which hydrophobic matter is deposited or printed and a plurality of spaced apart depressed regions. In a preferred embodiment, the hydrophobic matter applied to the elevated regions of the basesheet comprises hydrophobic fibers in a contiguous nonwoven web which has been apertured or provided with slits or other openings, such that the apertures or openings overlay a portion of the depressed regions. The elevated hydrophobic regions enhance dry feel and promote fluid flow toward the lower hydrophilic regions, which comprise the exposed depressed regions of the basesheet. The basesheet is preferably in liquid communication with underlying absorbent material, most preferably a stabilized airlaid cellulosic material or compressed stabilized fluff such that the absorbent material can wick fluid out of the basesheet by capillary action. When soft, hydrophobic fibers are deposited on the elevated regions, the liner also has a soft, cloth-like feel in addition to a dry feel in use.

U.S. Pat. No. 6,120,488 to VanRijswijck et al. discloses an absorbent article, such as a diaper, containing cuffs and a topsheet with one or more skin care compositions disposed thereon. The skin care compositions are transferable to the wearer's skin by normal contact and/or wearer motion and/or body heat. The skin care compositions disclosed in the present invention are selected to maintain and/or improve the skin health of the wearer upon transfer during use, for example, to provide a skin protective barrier or a therapeutic benefit; to minimize the abrasion between the cuffs and skin in the area where the cuffs contact the wearer's skin, resulting in less skin irritation; to improve BM clean up on the skin, or to improve the barrier properties of the cuffs.

U.S. Pat. No. 5,817,079 to Bergquist et al. discloses in absorbent products, such as sanitary napkins, discreet areas of dry fibrous materials such as fluid-repellent materials are precisely placed in various planes within the product so as to provide barriers to bodily fluid leakage from the product. In a preferred embodiment, hydrophobic fibers are placed around the periphery of a central absorbent area of an absorbent product to discourage and/or prevent side or end leakage from the product.

U.S. Pat. No. 5,830,201 to George et al. discloses a flushable diaper for use on the body of an infant or adult. An envelope has inner and outer walls which are secured together at their outer periphery to form an inner cavity. The outer wall is comprised of a hydrophobic outer layer together with a hydrophilic inner layer. The inner wall is comprised of a hydrophobic outer layer together with a hydrophilic inner layer. The inner and outer walls are separated by an interior cavity which contains one or more containers. The container is comprised of either a single layer or a laminated wall which forms a chamber that encloses a charge of water. The laminated wall is comprised a hydrophobic inner laminate and a hydrophilic outer laminate. When manually ruptured, the containers release water into the double walled envelope, which causes disintegration of the hydrophilic inner layers of the double walls, along with the hydrophobic outer layers. An absorbent member is carried on the inner wall of the envelope, and the absorbent member comprises a fluid pervious cover which encloses an inner body that is comprised of a material which is sufficiently hydrophilic to absorb urine and other fluids from the body while maintaining structural integrity of the inner body and to also absorb water in an effective amount to cause the inner body to disintegrate into small pieces.

U.S. Pat. No. 5,885,266 to Chihani et al. discloses an absorbent article such as a diaper, an incontinence guard or a sanitary napkin includes a liquid-permeable outer sheet, a liquid-impermeable bottom sheet and an absorbent body placed therebetween. The outer sheet and the absorbent body are at least partially joined together with a hydrophilic glue.

EP 0 850 628 A1 to Wierlacher disposes a tri-dimensional disposable sanitary napkin having a body facing surface and a garment facing surface, a longitudinal symmetry plane, a front end edge and a rear end edge, and comprising a liquid pervious topsheet, a backsheet joined to said topsheet and an absorbent core intermediate the backsheet and the topsheet. The absorbent core has a front portion, a central portion and a rear portion, and comprises a longitudinally oriented ridge in the central and rear portion having a profile that provides for an increased body fit. The sanitary napkin is intended for direct attachment to the skin of the wearer and comprises an adhesive on the body facing surface.

EP 0 850 627 A1 to Hirsch relates to disposable absorbent articles capable of self shaping in use as sanitary napkins or pantiliners. In particular, the disclosure relates to such disposable absorbent articles which are worn by direct attachment to the skin of the wearer in the area were absorption of bodily liquids is desired and which are activated during use to adapt their shape to the negative three dimensional image of a wearer. The topical adhesive attachment of such articles needs to be secure and pleasing upon application and during use of such articles, yet cause no discomfort upon removal of the article. This is achieved by selecting the rheological characteristics of adhesives for such articles.

EP 0 850 626 A1 relates to disposable absorbent articles particularly sanitary napkins, pantiliners, adult incontinence products or sweat pads. In particular, the disclosure relates to such disposable absorbent articles which are worn by direct attachment to the skin of the wearer in the area were absorption of bodily liquids is desired. The topical adhesive attachment of such articles needs to be secure and pleasing upon application and during use of such articles, yet cause no discomfort upon removal of the article. It is disclosed that in order to provide additional comfort the articles are adaptable, and preferably elastically adaptable.

European Patent 0 850 625 A1 relates to disposable absorbent articles particularly sanitary napkins, pantiliners, adult incontinence products which have side cuffs or baby diapers. In particular, the disclosure relates to such disposable absorbent articles with side cuffs which are maintained in their in use position by direct attachment to the skin of the wearer. The topical adhesive attachment of such side cuffs needs to be secure and pleasing upon application and during use of such articles, yet cause no discomfort upon removal of the article. This is achieved by selecting the rheological characteristics of adhesives for such articles.

European Patent 0 850 624 A1 relates to disposable absorbent articles particularly sanitary napkins, pantiliners, adult incontinence products or sweat pads. In particular, the disclosure relates to such disposable absorbent articles which are worn by direct attachment to the skin of the wearer in the area were absorption of bodily liquids is desired.

EP 0 850 623 A1 relates to disposable absorbent articles particularly sanitary napkins, pantiliners, adult incontinence products or sweat pads. In particular, the disclosure relates to such disposable absorbent articles which are worn by direct attachment to the skin of the wearer in the area were absorption of bodily liquids is desired and which articles are provided with an odor control system.

EP 0 850 622 A1 relates to breathable absorbent articles particularly sanitary napkins, pantiliners, adult incontinence products or sweat pads. In particular, the disclosure relates to such breathable absorbent articles which are worn by direct attachment to the skin of the wearer in the area were absorption of bodily liquids is desired.

EP 0 850 621 A1 relates to disposable absorbent articles particularly sanitary napkins, pantiliners, adult incontinence products or sweat pads. In particular, the disclosure relates to such disposable absorbent articles which are worn by direct attachment to the skin of the wearer in the area were absorption of bodily liquids is desired. According to the disclosure, in order to provide the articles with additional comfort they are adaptable, and preferably elastically adaptable.

EP 0 850 620 A1 relates to disposable absorbent articles particularly sanitary napkins, pantiliners, adult incontinence products which have side cuffs or baby diapers. In particular, the disclosure relates to such disposable absorbent articles with side cuffs which are maintained in their in use position by direct attachment to the skin of the wearer.

EP 0 850 619 A1 relates to disposable absorbent articles particularly sanitary napkins, pantiliners, adult incontinence products or sweat pads. In particular, the disclosure relates to such disposable absorbent articles which are worn by direct attachment to the skin of the wearer in the area were absorption of the bodily liquids is desired.

EP 0 850 618 A1 relates to absorbent articles capable of self shaping in use, particularly sanitary napkins or pantiliners. In particular, the disclosure relates to such disposable absorbent articles which are worn by direct attachment to the skin of the wearer in the area were absorption of bodily liquids is desired and which are activated during use to adapt to their shape to the negative three dimensional image of a wearer.

WO 00/07636 relates to disposable absorbent articles such as diapers and sanitary napkins which are provided with adhesive for attachment of the article to the skin which adhesives provide secure attachment and are pleasing to the skin upon application, yet cause no discomfort upon removal.

WO 98/28024 relates to topical adhesives for attachment to the skin. In particular, the disclosure relates to such topical adhesives which can be employed for attachment to the skin of protective articles, clothing, prosthesis, heat wraps, pads, and/or packs, e.g. for topical relief of pain or simply to provide warming; cold wraps, hearing aids, protective face masks, ornamental articles, or eye wear but excluding absorbent articles. The topical adhesives provides secure attachment and is pleasing to the skin upon application, yet causes no discomfort upon removal. This is achieved by selecting the chemical composition and rheological characteristics of the topical adhesives.

WO 98/28021 relates to topical adhesives for attachment to the skin. In particular, the disclosure relates to such topical adhesives which can be employed for attachment to the skin of articles such as protective articles, clothing, prothesis, heat wraps, pads, and/or packs, e.g. for topical relief of pain or simply to provide warmth; cold wraps, hearing aids, protective face masks, ornamental articles or eye wear, but excluding absorbent articles, or also of functional articles such as cosmetic or pharmaceutical delivery articles that provide a substance to the skin, decorative cosmetics or cleaning articles. The topical adhesive provides secure attachment and is pleasing to the particularly the viscous modulus G″ in combination with the thickness C of the topical adhesive layer in which the adhesive is provided for attachment to the skin.

WO 98/28017 relates to disposable absorbent articles particularly sanitary napkins, pantiliners, adult incontinence products which have side cuffs or baby diapers. In particular, the disclosure relates to such disposable absorbent articles with side cuffs which are maintained in their in-use position by direct attachment to the skin of the wearer.

WO 98/28016 relates to topical adhesives for attachment to the skin. In particular, the disclosure relates to such topical adhesives which can be employed for attachment to the skin of protective articles, clothing, prosthesis, heat wraps, pads, and/or packs, cold wraps, hearing aids, protective face masks, ornamental articles, or eye wear but excluding absorbent articles. The topical adhesive provides secure attachment and is pleasing to the skin upon application, yet causes no discomfort and a low level of adhesive residues on the skin upon removal. This is achieved by selecting the chemical composition and rheological characteristics of the topical adhesives, in particular with reference to the relationship between the elastic modulus G′ and the viscous modulus G″ of the adhesive.

WO 98/28014 relates to topical adhesives for attachment to the skin. In particular, the disclosure relates to such topical adhesives which can be employed for attachment to the skin in the area where absorption of bodily liquids is desired. The topical adhesive provides secure attachment and is pleasing to the skin upon application, yet causes no discomfort upon removal. This is achieved by selecting the chemical composition and rheological characteristics of the topical adhesives, particularly the viscous modulus G″ in combination with the thickness C of the topical adhesive layer applied to the absorbent article for attachment to the skin.

WO 98/27918 relates to disposable absorbent articles particularly sanitary napkins, pantiliners, adult incontinence products or sweat pads. In particular, the disclosure relates to such disposable absorbent articles which are worn by direct attachment to the skin of the wearer in the area where absorption of bodily liquids is desired. The topical adhesive attachment of such articles needs to be secure and pleasing upon application and during use of such articles, yet cause no discomfort upon removal of the article. This is achieved by selecting the rheological characteristics of adhesives for such articles.

WO 98/27917 relates to disposable articles, particularly sanitary napkins, pantiliners, adult incontinence products which have side cuffs, or baby diapers. In particular, the disclosure relates to such disposable absorbent articles with side cuffs which are maintained in their in-use position by direct attachment to the skin of the wearer. The topical adhesive attachment of such side cuffs needs to be secure and pleasing upon application and during use of such articles, yet cause no discomfort upon removal of the article. This is achieved by selecting the rheological characteristics of adhesives for such articles.

WO 98/27916 relates to disposable absorbent articles capable of self-shaping in use, particularly sanitary napkins or pantiliners. In particular, the disclosure relates to such disposable absorbent articles which are worn by direct attachment to the skin of the wearer where absorption of bodily liquids is desired and which are activated during use to adapt their shape to the negative three-dimensional image of a wearer. The topical adhesive attachment of such articles needs to be secure and pleasing upon application and during use of such articles, yet cause no discomfort upon removal of the article. This is achieved by selecting the rheological characteristics of adhesives for such articles.

WO 98/27915 relates to disposable articles particularly sanitary napkins, pantiliners, adult incontinence products or sweat pads. In particular, the disclosure relates to such disposable absorbent articles which are worn by direct attachment to the skin of the wearer in the area where absorption of bodily liquids is desired. The topical adhesive attachment of such articles needs to be secure and pleasing upon application and during use of such articles, yet cause no discomfort upon removal of the article.

WO 98/27913 relates to disposable absorbent articles capable of self shaping in use particularly sanitary napkins or pantiliners. Specifically disclosed are disposable absorbent articles which are worn by direct attachment to the skin of the wearer in the area where absorption of bodily liquids is desired and which are activated during use to adapt their shape to the negative three dimensional image of a wearer.

WO 98/27912 relates to disposable absorbent articles particularly sanitary napkins, pantiliners, adult incontinence products or sweat pads. The disclosure relates to such disposable absorbent articles which are worn by direct attachment to the skin of the wearer in the area where absorption of bodily liquids is desired and which are provided with an odour control system.

WO 98/27911 relates to breathable absorbent articles particularly sanitary napkins, pantiliners, adult incontinence products or sweat pads. Disclosed are breathable absorbent articles which are worn by direct attachment to the skin of the wearer in the area where absorption of bodily liquids is desired.

WO 98/27910 relates to disposable absorbent articles particularly sanitary napkins, pantiliners, adult incontinence products or sweat pads. In particular, the disclosure relates to such disposable absorbent articles which are worn by direct attachment to the skin of the wearer in the area where absorption of bodily liquids is desired.

WO 98/23305 relates to a polysiloxane adhesive composition that is disclosed as being useful in attaching products to human skin. It is disclosed that a disposable absorbent product may be prepared that includes the polysiloxane adhesive composition and that may be adhered directly to human skin and peeled off after use with little or no pain to the wearer.

As is apparent from the foregoing, each of the above discussed references presents a variety of means for improving fit and comfort of certain types of absorbent articles, as well as for controlling leakage in certain absorbent garments. However, all of these proposed means are deficient in terms of effectiveness and low product quality, mechanical complexity in design, specificity of application and/or associated cost inefficiencies.

In view of the deficiencies of the various products and processes disclosed in the above discussed references, it is highly desirable to provide cost-efficient absorbent articles that display superior fit, distribution, comfort, leak protection, skin protection, absorbency and/or cost-efficiency. Further, it is highly desirable to provide a cost-efficient process for producing absorbent articles having superior fit, distribution, comfort, leak protection, skin protection, absorbency and/or cost-efficiency, as well as having broad applicability.

### SUMMARY OF THE INVENTION

The present invention is directed to absorbent articles and absorbent garments, as well as methods for preparing same and methods for using same, that provide unexpectedly superior comfort to a wearer, as well as unexpectedly superior fit and consequentially superior leak protection. In particular, the absorbent articles and garments of the present invention comprise a polymer barrier and leg cuffs in a novel configuration. The present absorbent articles and absorbent garments are also highly cost-efficient.

An embodiment of the present invention provides an absorbent article comprising: a substantially impermeable backsheet; a permeable topsheet; an absorbent core disposed between the substantially impermeable backsheet and the permeable topsheet; a pair of elastic leg cuffs comprising a soft nonwoven material, each of said leg cuffs being disposed adjacent to the longitudinal side edges of the absorbent article; and a polymer barrier disposed between the substantially impermeable backsheet and the absorbent core, said polymer barrier being transversely positioned between the leg cuffs, such that the transverse width of the polymer barrier is at least the transverse width of the absorbent core and the transverse width of the polymer barrier is no greater than the distance between the innermost portion of each of the leg cuffs.

A further embodiment of the present invention provides an absorbent garment comprising: front and rear waist portions cooperating to form a waist opening; a crotch region formed between said front and rear waist portions; a pair of leg openings on opposed sides of the crotch region; a permeable topsheet; a substantially impermeable backsheet; an absorbent core disposed between the permeable topsheet and the substantially impermeable backsheet; a pair of stand-up elasticized leg gathers having a distal edge and a proximal edge and positioned inward of said leg openings on opposite sides of a longitudinal center line of the absorbent garment, said leg gathers comprising a non-woven material; a pair of elasticized leg cuffs comprising a soft nonwoven material, each of said leg cuffs being disposed adjacent to the longitudinal side edges of the absorbent article; and a polymer barrier disposed between the substantially impermeable backsheet and the absorbent core, said polymer barrier being transversely positioned between the leg cuffs, such that the transverse width of the polymer barrier is at least the transverse width of the absorbent core and the transverse width of the polymer barrier is no greater than the distance between the innermost portion of each of the leg cuffs.

An even further embodiment of the present invention provides an absorbent garment comprising: front and rear waist portions cooperating to form a waist opening; a crotch region formed between said front and rear waist portions; a pair of leg openings on opposed sides of the crotch region; a permeable topsheet, a substantially impermeable backsheet, and an absorbent core positioned between said permeable topsheet and said substantially impermeable backsheet; a pair of elasticized leg cuffs comprising a soft nonwoven material, each of said leg cuffs extending at least through the crotch region adjacent to each leg opening and positioned between said permeable topsheet and substantially impermeable backsheet; and a polymer barrier disposed between the substantially impermeable backsheet and the absorbent core, said polymer barrier being transversely positioned between the leg cuffs, such that the transverse width of the polymer barrier is at least the transverse width of the absorbent core and the transverse width of the polymer barrier is no greater than the distance between the innermost portion of each of the leg cuffs.

### BRIEF DESCRIPTION OF THE DRAWINGS

FIG. 1 is a top view of an absorbent article having a polymer barrier in accordance with an implementation of the present invention.

FIG. 2 is a cross-sectional view of an absorbent article having a polymer barrier in accordance with the implementation of the present invention illustrated in FIG. 1.

FIG. 3 is a top view of an absorbent article having a polymer barrier in accordance with an implementation of the present invention.

FIG. 4 is a cross-sectional view of an absorbent article having a polymer barrier in accordance with the implementation of the present invention illustrated in FIG. 3.

FIG. 5 is a top view of an absorbent article having a polymer barrier in accordance with an implementation of the present invention.

FIG. 6 is a cross-sectional view of an absorbent article having a polymer barrier in accordance with the implementation of the present invention illustrated in FIG. 5.

### DETAILED DESCRIPTION OF THE INVENTION

As used herein, the term “absorbent article” refers to articles that absorb and contain exudates, and more specifically refers to articles which are placed against or in proximity to the body of a wearer of the absorbent article to absorb and contain various exudates discharged from the body. A non-exhaustive list of examples of absorbent articles includes diapers, diaper cores, diaper covers, disposable diapers, training pants, feminine hygiene products and adult incontinence products. The term “disposable article” refers to absorbent articles that are intended to be discarded or partially discarded after a single use, i.e., they are not intended to be laundered or otherwise restored or reused. The term “unitary disposable absorbent article” refers to a disposable absorbent article that is essentially a single structure (i.e., it does not require separate manipulative parts such as a diaper cover and insert). As used herein, the term “diaper” refers to an absorbent article generally worn by infants and incontinent persons about the lower torso.

The claims are intended to cover all of the forgoing classes of absorbent articles, without limitation, whether disposable, unitary or otherwise. These classifications are used interchangeably throughout the specification, but are not intended to limit the claimed invention. The invention will be understood to encompass, without limitation, all classes of absorbent articles, including those described above. Preferably, the absorbent core is thin in order to improve the comfort and appearance of a garment. The employance of thin, comfortable garments is disclosed, for example without limitation in U.S. Pat. No. 5,098,423 to Pineiak et al. which is herein incorporated by reference.

The present invention provides an absorbent article having unexpectedly superior properties of absorbency, leakage protection and/or skin wellness, as well as a method of preparing and a method of using the absorbent article. In particular, the present invention is directed to an absorbent article comprising: a substantially impermeable backsheet; a permeable topsheet; an absorbent core disposed between the substantially impermeable backsheet and the permeable topsheet; a pair of elastic leg cuffs comprising a soft nonwoven material, each of said leg cuffs being disposed adjacent to the longitudinal side edges of the absorbent article; and a polymer barrier disposed between the substantially impermeable backsheet and the absorbent core, said polymer barrier being transversely positioned between the leg cuffs, such that the transverse width of the polymer barrier is at least the transverse width of the absorbent core and the transverse width of the polymer barrier is no greater than the distance between the innermost portion of each of the leg cuffs.

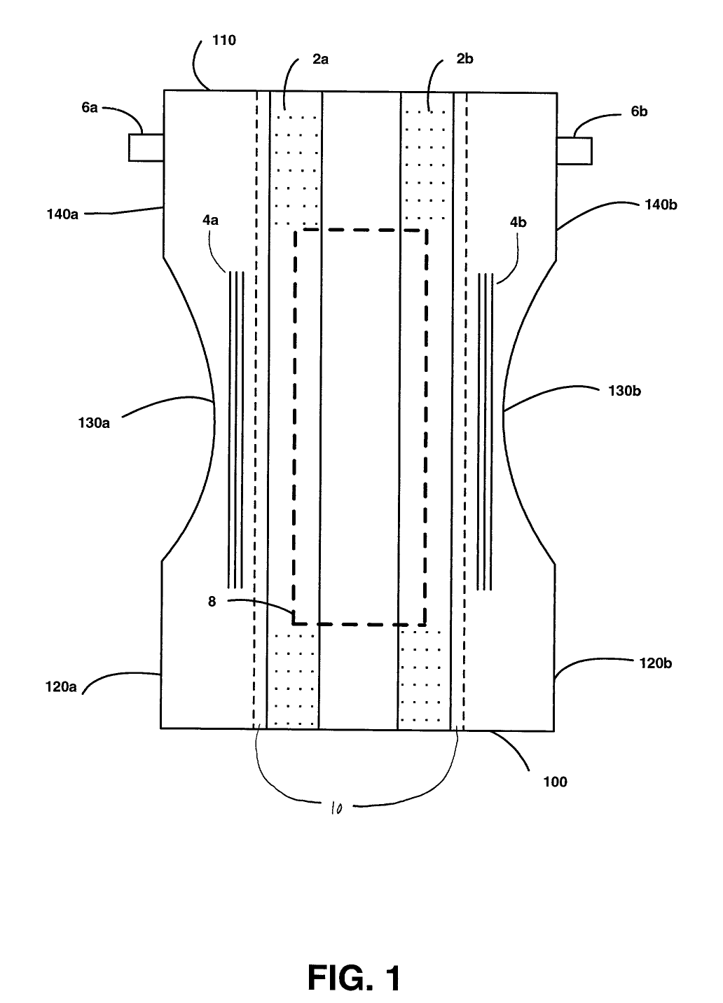

Referring to FIG. 1, an absorbent article is shown from a top perspective with the skin-facing surface of the permeable topsheet 40 and the skin-facing surface of the substantially impermeable backsheet 50 visible. The absorbent article comprises a front edge 100, a back edge 110, two front-side edges 120a and 120b, two back-side edges 140a and 140b, and two leg cutout edges 130a and 130b, positioned such that leg cutout edge 130a is disposed between front-side edge 120a and back-side edge 140a, and leg cutout edge 130b is disposed between front-side edge 120b and back-side edge 140b. The absorbent article further comprises two inner leg gathers 2a and 2b, each of which extends longitudinally along the topsheet of the absorbent article from the back edge 110 to the front edge 100. The inner leg gathers 2a and 2b are approximately parallel to one another and are each positioned approximately parallel to the front-side and back-side edges 120a, 120b, 140a and 140b. Further, each inner leg gather is located adjacent to and inward of the inner most portion of the leg cutout edges 130a and 130b. A pair of front wing regions are defined as the area between each front-side edge and its adjacent inner leg gather, forward of the leg cutouts. A pair of back wing regions are defined as the area between adjacent back-side edge and its adjacent inner leg gather, rearward of the leg cutouts.

As further shown in FIG. 1, the absorbent article further comprises two leg cuffs 4a and 4b, each of which is positioned approximately adjacent and parallel to each of the inner leg gathers 2a and 2b. Each of the leg cuffs is located outward of said inner leg gathers 2a and 2b. Two fasteners 6a and 6b are each located at or near the back side edge of the absorbent article. An absorbent core 8 is shown as being disposed between the permeable topsheet 40 and a substantially impermeable backsheet 50 (not shown in FIG. 1). The absorbent core 8 is positioned longitudinally along the absorbent article. Waist foam is optionally present in the absorbent article and positioned near and adjacent to the front edge 100 and/or back edge 110, on the skin-facing surface of the permeable topsheet 40.

Referring still to FIG. 1, a polymer barrier 10 is shown. The polymer barrier 10 is disposed between the permeable topsheet 40 and the substantially impermeable backsheet 50. On the portion of the absorbent article corresponding to the absorbent core 8, the polymer barrier 10 is disposed between the absorbent core 8 and the substantially impermeable backsheet 50. The polymer barrier 10 is transversely positioned between the leg cuffs 4a and 4b and extends from about the front edge 100 to about the back edge 110. The inner leg gathers 2a and 2b are disposed on the skin-facing surface of the permeable topsheet 40 and are bonded thereto.

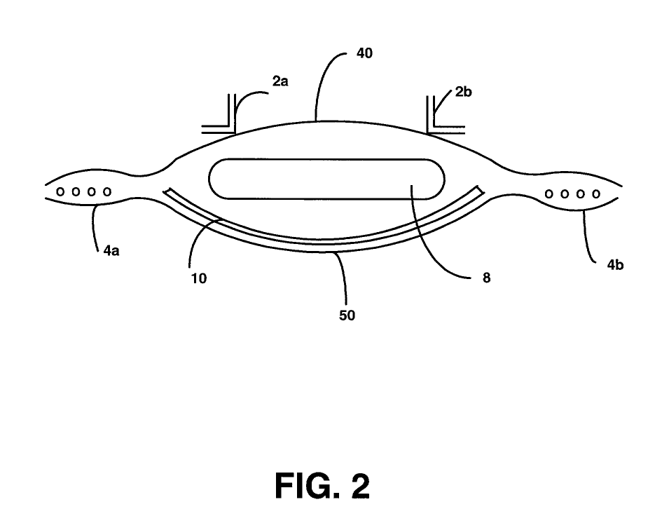

Referring to FIG. 2, a cross-sectional view of an absorbent article according to the implementation of the present invention show in FIG. 1 is shown. The polymer barrier 10 is shown disposed between the substantially impermeable backsheet 50 and the permeable topsheet 40. Further, the polymer barrier 10 is shown as being disposed between the absorbent core 8 and the substantially impermeable backsheet 50 at the portion of the polymer barrier 10 that corresponds to the absorbent core 8.

Referring still to FIG. 2, the leg cuffs 4a and 4b are shown as being positioned outward of the polymer barrier 10 and along the permeable topsheet 40 and the substantially impermeable backsheet 50. In this manner, the leg cuffs 4a and 4b are bonded to the permeable topsheet 40 and/or the substantially impermeable backsheet 50. Accordingly, as illustrated in the figure, the leg cuffs 4a and 4b are not bonded to the polymer barrier 10. Thus, the leg cuffs 4a and 4b comprise the softer material of the permeable topsheet 40 and/or the substantially impermeable backsheet 50 rather than the stiffer polymer material of the polymer barrier 10.

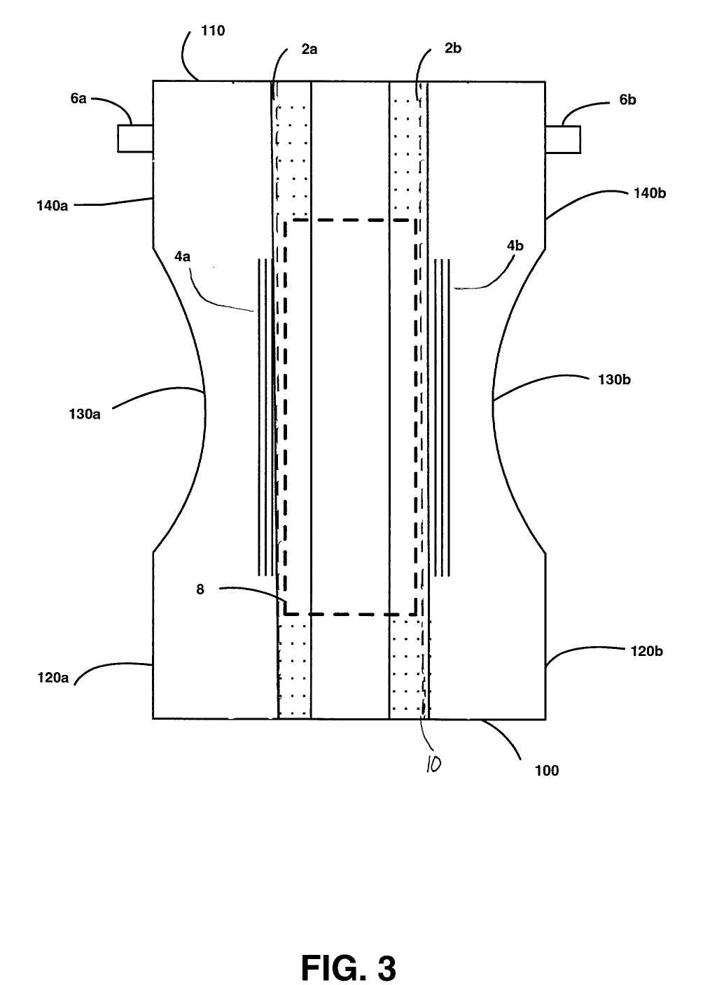

Referring to FIG. 3, an absorbent article is shown from a top perspective with the skin-facing surface of the permeable topsheet 40 and the skin-facing surface of the substantially impermeable backsheet 50 visible. The absorbent article comprises a front edge 100, a back edge 110, two front-side edges 120a and 120b, two back-side edges 140a and 140b, and two leg cutout edges 130a and 130b, positioned such that leg cutout edge 130a is disposed between front-side edge 120a and back-side edge 140a, and leg cutout edge 130b is disposed between front-side edge 120b and back-side edge 140b. The absorbent article further comprises two inner leg gathers 2a and 2b, each of which extends longitudinally along the topsheet of the absorbent article from the back edge 110 to the front edge 100. The inner leg gathers 2a and 2b are approximately parallel to one another and are each positioned approximately parallel to the front-side and back-side edges 120a, 120b, 140a and 140b. Further, the innermost portion of each inner leg gather is located adjacent to and inward of the innermost portion of the leg cutout edges 130a and 130b. The outermost portion of each of the inner leg gathers 2a and 2b extends beyond the outermost portion of the permeable topsheet 40 and at least to the nearest leg cuff 4a or 4b. Further, a pair of front wing regions are defined as the area between each front-side edge and its adjacent inner leg gather, forward of the leg cutouts. A pair of back wing regions are defined as the area between adjacent back-side edge and its adjacent inner leg gather, rearward of the leg cutouts.

As further shown in FIG. 3, the absorbent article further comprises two leg cuffs 4a and 4b, each of which is positioned approximately adjacent and parallel to each of the inner leg gathers 2a and 2b. Each of the leg cuffs is located outward of said inner leg gathers 2a and 2b. Two fasteners 6a and 6b are each located at or near the back side edge of the absorbent article. An absorbent core 8 is shown as being disposed between the permeable topsheet 40 and a substantially impermeable backsheet 50 (not shown in FIG. 1). The absorbent core 8 is positioned longitudinally along the absorbent article. Waist foam is optionally present in the absorbent article and positioned near and adjacent to the front edge 100 and/or back edge 110, on the skin-facing surface of the permeable topsheet 40.

Referring still to FIG. 3, a polymer barrier 10 is shown. The polymer barrier 10 is disposed between the permeable topsheet 40 and the substantially impermeable backsheet 50. On the portion of the absorbent article corresponding to the absorbent core 8, the polymer barrier 10 is disposed between the absorbent core 8 and the substantially impermeable backsheet 50. The polymer barrier 10 is transversely positioned between the leg cuffs 4a and 4b and extends from about the front edge 100 to about the back edge 110. The inner leg gathers 2a and 2b are disposed on the skin-facing surface of the permeable topsheet 40 and are bonded thereto.

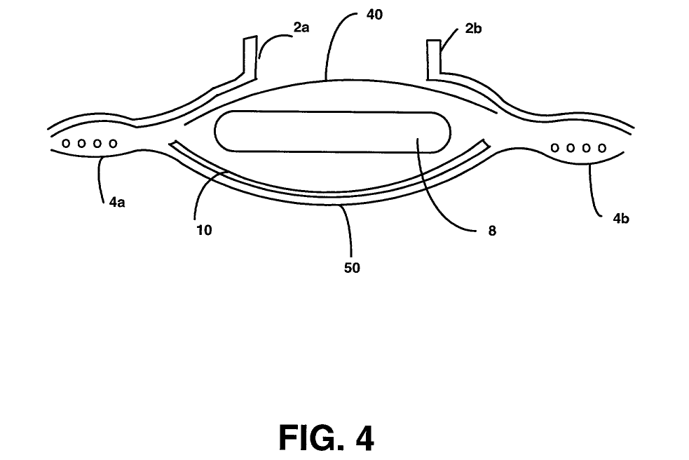

Referring to FIG. 4, a cross-sectional view of an absorbent article according to the implementation of the present invention show in FIG. 1 is shown. The polymer barrier 10 is shown disposed between the substantially impermeable backsheet 50 and the permeable topsheet 40. Further, the polymer barrier 10 is shown as being disposed between the absorbent core 8 and the substantially impermeable backsheet 50 at the portion of the polymer barrier 10 that corresponds to the absorbent core 8.

Referring still to FIG. 4, the leg cuffs 4a and 4b are shown as being positioned outward of the polymer barrier 10 and along each inner leg gather 2a and 2b and the substantially impermeable backsheet 50. In this manner, the leg cuffs 4a and 4b are bonded to a portion of each of the inner leg gathers 2a and 2b and/or the substantially impermeable backsheet 50. Accordingly, as illustrated in the figure, the leg cuffs 4a and 4b are not bonded to the polymer barrier 10. Thus, the leg cuffs 4a and 4b comprise the softer material of the inner leg gathers 2a and 2b and/or the substantially impermeable backsheet 50 rather than the stiffer polymer material of the polymer barrier 10.

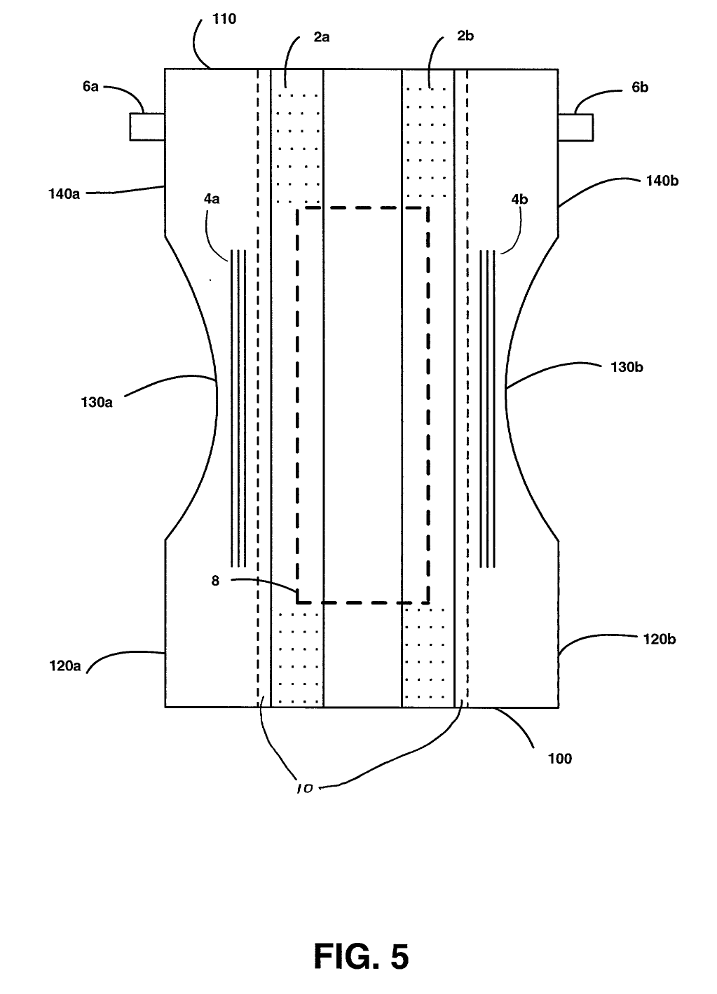

Referring to FIG. 5, an absorbent article is shown from a top perspective with the skin-facing surface of the permeable topsheet 40 and the skin-facing surface of the substantially impermeable backsheet 50 visible. The absorbent article comprises a front edge 100, a back edge 110, two front-side edges 120a and 120b, two back-side edges 140a and 140b, and two leg cutout edges 130a and 130b, positioned such that leg cutout edge 130a is disposed between front-side edge 120a and back-side edge 140a, and leg cutout edge 130b is disposed between front-side edge 120b and back-side edge 140b. The absorbent article further comprises two inner leg gathers 2a and 2b, each of which extends longitudinally along the permeable topsheet 40 of the absorbent article from the back edge 110 to the front edge 100. The inner leg gathers 2a and 2b are approximately parallel to one another and are each positioned approximately parallel to the front-side and back-side edges 120a, 120b, 140a and 140b. Further, each inner leg gather is located adjacent to and inward of the inner most portion of the leg cutout edges 130a and 130b. A pair of front wing regions are defined as the area between each front-side edge and its adjacent inner leg gather, forward of the leg cutouts. A pair of back wing regions are defined as the area between adjacent back-side edge and its adjacent inner leg gather, rearward of the leg cutouts.

As further shown in FIG. 5, the absorbent article further comprises two leg cuffs 4a and 4b, each of which is positioned approximately adjacent and parallel to each of the inner leg gathers 2a and 2b. Each of the leg cuffs is located outward of said inner leg gathers 2a and 2b. Two fasteners 6a and 6b are each located at or near the back side edge of the absorbent article. An absorbent core 8 is shown as being disposed between the permeable topsheet 40 and a substantially impermeable backsheet 50 (not shown in FIG. 1). The absorbent core 8 is positioned longitudinally along the absorbent article. Waist foam is optionally present in the absorbent article and positioned near and adjacent to the front edge 100 and/or back edge 110, on the skin-facing surface of the permeable topsheet 40.

Referring still to FIG. 5, a polymer barrier 10 is shown. The polymer barrier 10 is disposed between the permeable topsheet 40 and the substantially impermeable backsheet 50. On the portion of the absorbent article corresponding to the absorbent core 8, the polymer barrier 10 is disposed between the absorbent core 8 and the substantially impermeable backsheet 50. The polymer barrier 10 is transversely positioned between the leg cuffs 4a and 4b and extends from about the front edge 100 to about the back edge 110. The inner leg gathers 2a and 2b are disposed on the skin-facing surface of the permeable topsheet 40 and are bonded thereto.

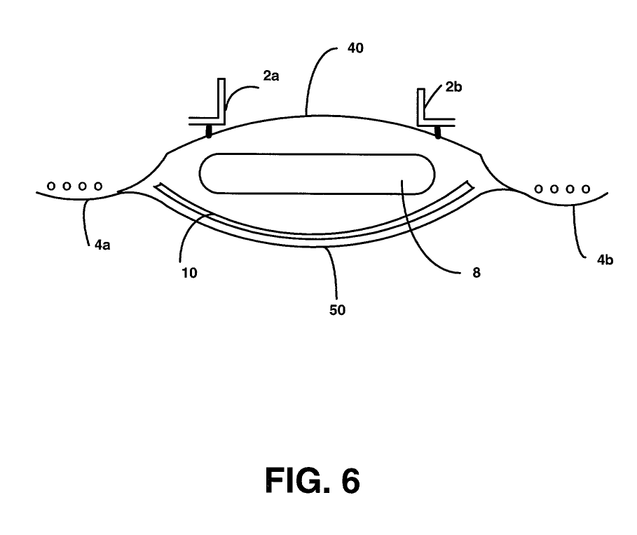

Referring to FIG. 6, a cross-sectional view of an absorbent article according to the implementation of the present invention show in FIG. 1 is shown. The polymer barrier 10 is shown disposed between the substantially impermeable backsheet 50 and the permeable topsheet 40. Further, the polymer barrier 10 is shown as being disposed between the absorbent core 8 and the substantially impermeable backsheet 50 at the portion of the polymer barrier 10 that corresponds to the absorbent core 8.

Referring still to FIG. 6, the leg cuffs 4a and 4b are shown as being positioned outward of the polymer barrier 10 and about the substantially impermeable backsheet 50. In this manner, the leg cuffs 4a and 4b are bonded to the substantially impermeable backsheet 50. Accordingly, as illustrated in the figure, the leg cuffs 4a and 4b are not bonded to the polymer barrier 10. Thus, the leg cuffs 4a and 4b comprise the softer material (i.e., soft nonwoven material) of the substantially impermeable backsheet 50 rather than the stiffer polymer material of the polymer barrier 10.

The novel configuration illustrated in the figures herein unexpectedly provides protection against leakage directly through the substantially impermeable backsheet while dramatically improving comfort for the wearer. A further unexpected benefit of the novel configuration is that the softer material provides for an improved fit about the legs of the wearer, thereby providing unexpectedly superior lateral leak protection (e.g., protection against leakage at the leg openings). This further enhances comfort to the wearer, as well as providing skin wellness benefits to the wearer. Moreover, the absorbent articles in accordance with the present invention are highly cost-efficient in addition to providing the foregoing superior characteristics.

According to an implementation of the present invention, an absorbent article comprises: a substantially impermeable backsheet; a permeable topsheet; an absorbent core disposed between the substantially impermeable backsheet and the permeable topsheet; a pair of leg cuffs, each of said leg cuffs being disposed adjacent to the longitudinal side edges of the absorbent article; and a polymer barrier disposed between the substantially impermeable backsheet and the absorbent core, said polymer barrier being transversely positioned between the leg cuffs, such that the transverse width of the polymer barrier is at least the transverse width of the absorbent core and the transverse width of the polymer barrier is no greater than the distance between the innermost portion of each of the leg cuffs.

Suitable leg cuffs include any leg cuffs that hold the leg openings of the absorbent article or garment snug to the legs of a wearer of the absorbent article or garment. The leg cuffs may be elastic leg cuffs or the like. The leg cuffs comprise a soft nonwoven material. Optionally, the leg cuffs are directly bonded to the substantially impermeable backsheet, the permeable topsheet and/or the inner leg gathers and any such combination.

The soft nonwoven material comprises fibers that are sufficiently fine to feel comfortable against the skin of a wearer during use, including during prolonged or extended use, ranging from perhaps a few hours to an entire evening and nighttime. The fineness of the fibers in the soft nonwoven material ranges from about 0.90 dtex to about 4.5 dtex. Preferably, the fineness of the fibers is about 1.00 dtex to about 3.50 dtex. More preferably, the fineness of the fibers is about 1.25 dtex to about 3.00 dtex. Even more preferably, the fineness of the fibers is about 1.25 dtex to about 2.75 dtex. The soft nonwoven material may comprise a variety of suitable fibers effective in implementations of the present invention. A person skilled in the art would readily be able to select a suitable soft nonwoven material in accordance with the present invention.

Suitable soft nonwoven materials may be prepared using a variety of methods and techniques well known in the art. For example, soft nonwoven materials may be prepared using conventional processes such as spun-bonding, melt-blowing, heat rolling, suction heat bonding and air-through bonding, without limitation.

Non-limiting exemplary fibers include spun-laced fibers, spun-bonded fibers, melt-blown fibers and combinations thereof. The soft nonwoven material may be of a variety of suitable substances. Non-limiting exemplary nonwoven materials include polyester, polypropylene, polyethylene and combinations thereof. Persons of ordinary skill in the art would readily be able to select and/or prepare suitable fibers in accordance with implementations of the present invention using conventional skills and techniques, based upon the guidance provided herein.

The substantially impermeable backsheet may comprise a nonwoven layer. The nonwoven layer may comprise a variety of suitable materials. Preferably, the nonwoven layer is a soft nonwoven material or a combination of soft nonwoven materials, without limitation. More preferably, the nonwoven layer is a soft nonwoven material or combinations thereof and the soft nonwoven material comprises fibers selected from the group consisting of polyester fibers, polypropylene fibers, polyethylene fibers and combinations thereof, without limitation.

The absorbent article and/or absorbent garment is configured so that the polymer barrier provides adequate leak protection. For example, the longitudinal side edges of the polymer barrier are positioned at an effective distance from the adjacent longitudinally disposed side edges of the absorbent core to provide protection from leakage through the substantially impermeable backsheet while avoiding contact with the legs of the wearer during use. A person skilled in the art would readily be able to select a suitable effective distance in accordance with the present invention.

The absorbent article and/or absorbent garment is further configured so that the polymer barrier does not come into contact with the legs of the wearer of the absorbent article and/or garment during use. For example, each longitudinal side edge of the polymer barrier is positioned an effective distance from the inner edge of each leg cuff. A person skilled in the art would readily be able to select a suitable effective distance in accordance with the present invention.

The absorbent core of the present invention may be of a variety of suitable shapes and sizes. Persons of ordinary skill in the art would be readily able to prepare suitable absorbent cores for the absorbent articles and garments of the present invention. Preferably, the absorbent core of the present invention is approximately rectangular.

The polymer barrier may be of a variety of shapes and sizes that are suitable for the absorbent articles and garments of the present invention, as would be recognized by persons of ordinary skill in the art. Preferably, the polymer barrier of the present invention is approximately rectangular, without limitation.

Optionally, the articles and garments of the present invention further comprise a pair of inner leg gathers disposed on the permeable topsheet, each of said inner leg gathers being positioned outward of a side edge of the absorbent core and inward of the nearest leg cuff. The transverse width of the polymer barrier may be less than, greater than or about equal to the transverse distance between the inner leg gathers. Preferably, the transverse width of the polymer barrier is greater than the transverse distance between the inner leg gathers.

According to an implementation of the present invention, an absorbent garment comprises: front and rear waist portions cooperating to form a waist opening; a crotch region formed between said front and rear waist portions; a pair of leg openings on opposed sides of the crotch region; a permeable topsheet; a substantially impermeable backsheet; an absorbent core disposed between the permeable topsheet and the substantially impermeable backsheet; a pair of stand-up elasticized leg gathers having a distal edge and a proximal edge and positioned inward of said leg openings on opposite sides of a longitudinal center line of the absorbent garment, said leg gathers comprising a non-woven material; a pair of leg cuffs, each of said leg cuffs being disposed adjacent to the longitudinal side edges of the absorbent article; and a polymer barrier disposed between the substantially impermeable backsheet and the absorbent core, said polymer barrier being transversely positioned between the leg cuffs, such that the transverse width of the polymer barrier is at least the transverse width of the absorbent core and the transverse width of the polymer barrier is no greater than the distance between the innermost portion of each of the leg cuffs.

A wide variety of permeable topsheets are suitable for the absorbent articles and garments of the present invention. For example, the permeable topsheet of the absorbent garment in accordance may have a basis weight of about 0.6 ounces per square yard, without limitation. Further, the permeable topsheet may comprise a central liquid permeable portion and a pair of side marginal portions joined to respective opposite side edges of said central portion, said side marginal portions comprising spun-bond, melt-blown, spun-bond (SMS) non-woven material having a basis weight in the range of about 0.3-0.8 ounces per square yard, said central portion of said topsheet comprising a spun-bond polypropylene non-woven fabric, without limitation.

The substantially impermeable backsheet comprises a non-woven material. The non-woven material may be of a wide variety of suitable materials as would be readily apparent to persons of ordinary skill in the art, based upon the disclosure provided herein. Preferably, the nonwoven material is selected from the group consisting of spun-laced fibers, spun-bonded, melt-blown fibers and combinations thereof, without limitation. More preferably, the nonwoven material comprises fibers of a material selected from the group consisting of polyester, polypropylene, polyethylene and combinations thereof, without limitation.

According to an implementation of the present invention, an absorbent garment comprises: front and rear waist portions cooperating to form a waist opening; a crotch region formed between said front and rear waist portions; a pair of leg openings on opposed sides of the crotch region; a permeable topsheet, a substantially impermeable backsheet, and an absorbent core positioned between said permeable topsheet and said substantially impermeable backsheet; a pair of leg cuffs, each of said leg cuffs extending at least through the crotch region adjacent to each leg opening and positioned between said permeable topsheet and substantially impermeable backsheet; and a polymer barrier disposed between the substantially impermeable backsheet and the absorbent core, said polymer barrier being transversely positioned between the leg cuffs, such that the transverse width of the polymer barrier is at least the transverse width of the absorbent core and the transverse width of the polymer barrier is no greater than the distance between the innermost portion of each of the leg cuffs.

In accordance with an implementation of the present invention, an absorbent article is substantially evenly distributed around the body of the wearer during use. Even distribution of the absorbent article is facilitated through the novel configuration of the polymer barrier and the leg cuffs. The even distribution improves fit and comfort of the absorbent article and consequently provides unexpectedly superior leakage protection.

Due to the wide variety of materials which may be incorporated into the absorbent articles of the present invention, the present invention is not intended to be limited to any specific materials. The permeable topsheet, substantially impermeable backsheet, absorbent core and other components of the absorbent articles in accordance with various implementations of the present invention may comprise various materials. Persons of ordinary skill in the art would be readily able to select appropriate materials for use in the various components of the present invention based upon the materials.

In accordance with various implementations of the present invention, the absorbent core may contain one or more fibers, one or more polymers or combinations thereof. Non-limiting exemplary fibers which may be used in the articles of the present invention include, without limitation, cellulose fibers, cellulose acetate fibers, rayon fibers, Courtauld's LYOCEL fibers, polyacrylonitrile fibers, surface modified (hydrophilic) polyester fibers, surface modified polyolophin/polyester by component fibers, surface modified polyester/polyester bicomponent fibers, cotton fibers or blends thereof. Preferably, cellulose acetate, rayon, Courtauld's LYOCEL, polyacrylonitrile, cotton fibers and cotton linters or combinations thereof are used in the present invention. More preferably, cellulose fibers are used as fiber material in the present invention.

Other materials may be added to fiber or pulp material which is optionally processed in a fiberizing apparatus, such as a hammermill. The additives may be added at any point in the process. Preferably, the additives are sprayed or injected into the airborne fibers prior to the depositing of the fibers on a forming surface. Non-limiting exemplary additives which may be incorporated into the process of the present invention include a polymer such as a super absorbent polymer (SAP), hydrophilic polymers, potato starch, corn starch, wheat starch or rice starch, or combinations thereof.

Various different combinations of materials may be used as are known to persons of ordinary skill in the art and which are described in U.S. Pat. No. 6,068,620 which is herein incorporated by reference. Preferably, the mixtures incorporated in the invention are substantially homogenous mixtures or uniformly distributed mixtures. Absorbent articles in accordance with an implementation of the present invention are prepared using conventional methods and materials well known to persons of ordinary skill in the art, using the guidelines provided herein.

The present invention further provides absorbent garments that include the absorbent article of the present invention. Non-limiting exemplary absorbent garments include diapers, adult incontinent products, feminine hygiene products, disposable sanitary products and the like. Non-limiting exemplary diapers include daytime diapers, nighttime diapers, daytime/nighttime diapers, long-term wear diapers, male diapers, female diapers, unisex diapers, travel diapers, medicated diapers, swimming diapers and the like.

The present invention has been described in connection with the preferred embodiments. These embodiments, however, are merely for example and the invention is not restricted thereto. Any examples described herein are illustrative of preferred embodiments of the inventive subject matter and are not to be construed as limiting the inventive subject matter thereto. It will be understood by persons skilled in the art that other variations and modifications can easily be made within the scope of the invention as defined by the appended claims.

What is claimed is:

1. An absorbent article comprising:
a substantially impermeable backsheet;
a permeable topsheet;
an absorbent core disposed between the substantially impermeable backsheet and the permeable topsheet;
a pair of elastic leg cuffs comprising a soft nonwoven material, each of said elastic leg cuffs being disposed adjacent to the longitudinal side edges of the absorbent article; and
a polymer barrier disposed between the substantially impermeable backsheet and the absorbent core, said polymer barrier being transversely positioned between the elastic leg cuffs, such that the transverse width of the polymer barrier is at least the transverse width of the absorbent core and the transverse width of the polymer barrier is no greater than the distance between the innermost portion of each of the elastic leg cuffs.

2. The absorbent article of claim 1, wherein the soft nonwoven material comprises fibers selected from the group consisting of spun-laced fibers, spun-bonded fibers, melt-blown fibers and combinations thereof.

3. The absorbent article of claim 1, wherein the soft nonwoven material comprises fibers of about 1.00 dtex to about 3.50 dtex.

4. The absorbent article of claim 1, wherein the soft nonwoven material comprises fibers of about 1.25 dtex to about 3.00 dtex.

5. The absorbent article of claim 1, wherein the soft nonwoven material comprises fibers of about 1.25 dtex to about 2.75 dtex..

6. The absorbent article of claim 1, wherein said soft nonwoven material has a basis weight in the range of about 0.3-0.8 ounces per square yard.

7. The absorbent article of claim 1, wherein the soft nonwoven material is selected from the group consisting of polyester, polypropylene, polyethylene and combinations thereof.

8. The absorbent article of claim 1, wherein the leg cuffs are directly bonded to the substantially impermeable backsheet.

9. The absorbent article of claim 1, wherein the leg cuffs are directly bonded to the substantially impermeable backsheet and the permeable topsheet.

10. The absorbent article of claim 1, wherein the absorbent core is approximately rectangular.

11. The absorbent article of claim 1, wherein the polymer barrier is approximately rectangular.

12. The absorbent article of claim 1, wherein the substantially impermeable backsheet comprises the soft nonwoven material 1.

13. The absorbent article of claim 1, wherein the soft nonwoven material is selected from the group consisting of polyester fibers, polypropylene fibers, polyethylene fibers and combinations thereof.

14. The absorbent article of claim 1, wherein the polymer barrier is positioned on said absorbent article so as to not come into contact with the legs of the wearer of the absorbent article during use.

15. The absorbent article of claim 1, further comprising a pair of inner leg gathers disposed on the permeable topsheet, each of said inner leg gathers being positioned outward of a side edge of the absorbent core and inward of the nearest leg cuff.

16. The absorbent article of claim 1, wherein the transverse width of the polymer barrier is greater than the transverse distance between each of the inner leg gathers.

17. An absorbent garment comprising
front and rear waist portions cooperating to form a waist opening;
a crotch region formed between said front and rear waist portions;
a pair of leg openings on opposed sides of the crotch region;
a permeable topsheet;
a substantially impermeable backsheet;
an absorbent core disposed between the permeable topsheet and the substantially impermeable backsheet; a pair of stand-up elasticized leg gathers having a distal edge and a proximal edge and positioned inward of said leg openings on opposite sides of a longitudinal center line of the absorbent garment, said leg gathers comprising a non-woven material;
a pair of elastic leg cuffs comprising a soft nonwoven material, each of said elastic leg cuffs being disposed adjacent to the longitudinal side edges of the absorbent article; and
a polymer barrier disposed between the substantially impermeable backsheet and the absorbent core, said polymer barrier being transversely positioned between the elastic leg cuffs, such that the transverse width of the polymer barrier is at least the transverse width of the absorbent core and the transverse width of the polymer barrier is no greater than the distance between the innermost portion of each of the elastic leg cuffs.

18. The absorbent garment of claim 17, said permeable topsheet having a basis weight of about 0.6 ounces per square yard, said permeable topsheet comprising a central liquid permeable portion and a pair of side marginal portions joined to respective opposite side edges of said central portion, said side marginal portions comprising spun-bond, melt-blown, spun-bond (SMS) non-woven material having a basis weight in the range of about 0.3-0.8 ounces per square yard, said central portion of said topsheet comprising a spun-bond polypropylene non-woven fabric.

19. The absorbent garment of claim 17, said substantially impermeable backsheet comprising a non-woven material selected from the group consisting of spun-laced fibers, spun-bonded, melt-blown fibers and combinations thereof.

20. The absorbent garment of claim 17, wherein said fibers comprise a material selected from the group consisting of polyester, polypropylene, polyethylene and combinations thereof.

21. The absorbent article of claim 17, wherein the polymer barrier is positioned on said absorbent article so as to not come into contact with the legs of the wearer of the absorbent article during use.

22. An absorbent garment comprising:
front and rear waist portions cooperating to form a waist opening;
a crotch region formed between said front and rear waist portions;
a pair of leg openings on opposed sides of the crotch region;
a permeable topsheet, a substantially impermeable backsheet, and an absorbent core positioned between said permeable topsheet and said substantially impermeable backsheet;
a pair of elastic leg cuffs comprising a soft nonwoven material, each of said elastic leg cuffs extending at least through the crotch region adjacent to each leg opening and positioned between said permeable topsheet and substantially impermeable backsheet; and
a polymer barrier disposed between the substantially impermeable backsheet and the absorbent core, said polymer barrier being transversely positioned between the elastic leg cuffs, such that the transverse width of the polymer barrier is at least the transverse width of the absorbent core and the transverse width of the polymer barrier is no greater than the distance between the innermost portion of each of the elastic leg cuffs.

23. The absorbent garment of claim 22, wherein the soft nonwoven material comprises fibers selected from the group consisting of spun-laced fibers, spun-bonded fibers, melt-blown fibers and combinations thereof.

24. The absorbent garment of claim 22, wherein the soft nonwoven material is selected from the group consisting of polyester, polypropylene, polyethylene and combinations thereof.

25. The absorbent article of claim 22, wherein the polymer barrier is positioned on said absorbent article so as to not come into contact with the legs of the wearer of the absorbent article during use.
